(19) United States
(12) Patent Application Publication
(10) Pub. No.: US 2008/0306310 A1
(43) Pub. Date: Dec. 11, 2008

(54) AVOIDANCE OF SOLIDS DEPOSITS IN DROPLET SEPARATORS BY METERING IN SUITABLE LIQUIDS

(75) Inventors: Christian MUENNICH, Leverkusen (DE); Ernst-Joachim ALPS, Dormagen (DE); Stefan WESTERNACHER, Kempen (DE)

Correspondence Address: CONNOLLY BOVE LODGE & HUTZ, LLP, P O BOX 2207, WILMINGTON, DE, 19899, US

(73) Assignee: BAYER MATERIALSCIENCE AG, LEVERKUSEN (DE)

(21) Appl. No.: 12/133,447

(22) Filed: Jun. 5, 2008

(30) Foreign Application Priority Data
Jun. 8, 2007 (DE) 102007026548.6

(51) Int. Cl. C07C 37/68

(52) U.S. Cl. 568/724

(57) ABSTRACT

The present invention relates to a process for the avoidance of solids deposits in droplet separators (demisters) by metering in suitable liquids, in particular in the production of bis(4-hydroxyaryl)alkanes.

### RELATED APPLICATIONS

This application claims benefit to German Patent Application No. 10 2007 026 548.6, filed Jun. 8, 2007, which is incorporated herein by reference in its entirety for all useful purposes.

### BACKGROUND OF THE INVENTION

The present invention relates to a method for avoiding solids deposits in droplet separators (demisters) by metering in (spraying in) suitable liquids in the production of bis(4-hydroxyaryl)alkanes.

The synthesis of bis(4-hydroxyaryl)alkanes by acid-catalysed reaction of aromatic hydroxy compounds with ketones is known, for example from U.S. Pat. No. 2,775,620 or EP-A 342 758. As a rule an adduct of the bis(4-hydroxyaryl)alkane and the aromatic monohydroxy compound used as educt is obtained as intermediate product, which is then removed from the aromatic hydroxy compound by distillation, for example as described in EP-A 343 349 or EP-A 1 121 339.

The separation of the aromatic monohydroxy compound from the melt, also termed “stripping”, from the bis(4-hydroxyaryl)alkane is known in the literature. In EP-A 343 349 the aromatic monohydroxy compound is separated from the bis(4-hydroxyaryl)alkane for example with steam in a packed column at 160° to 200° C. under slightly reduced pressure. The use of steam as stripping medium can lead to decomposition reactions of the bis(4-hydroxyaryl)alkane. The decomposition products thereby formed can adversely affect the quality of the products produced from the bisphenols, such as epoxy resins, polyesters, polyester carbonates and polycarbonates, and in particular can result in colour problems, poor light permeability of transparent products, or specks in the surfaces of moulded parts made from these end products.

EP-A 1 121 339 describes a separation of the phenol by an inert gas (nitrogen) that is less damaging to the product, and which compared to steam leads to less decomposition of the bis(4-hydroxyaryl)alkane. After separation of the phenol by condensation, the nitrogen is compressed, washed and, after preheating, recycled to the desorber.

In the scrubbing of the compressed nitrogen with water, fine droplets of the wash liquid remain in this gas stream and can lead to deposits, for example in the downstream heat exchanger that is used to preheat the nitrogen.

Accordingly, a demister can be used in the gas stream between the scrubbing stage and the heat exchanger, which performs the function of a droplet separator and thus prevents deposits in the downstream parts of the plant. This separation of droplets from the gas stream on the demister is however accompanied by a blockage of the demister itself due to deposits of phenolic constituents, which can come from the wash liquid of the wash stage or from an incomplete deposition in the wash step. These deposits lead to blockages and reduce the functional capability of the demister, and even cause production stoppages on account of cleaning work.

Demisters as droplet separators are already known and are described in “RÖMPP, Lexikon Chemie 2.0”. From WO-A 2006/106582 it is also known that in the deposition of (seawater) liquid droplets from gases on demisters, salt deposits can form on these demisters. In order to avoid such depositions it is proposed in WO-A 2006/106582 to construct the demister from deflector plates of a specific configuration so that slight deposits on these deflector plates of the demister are washed away by droplets running off the plates. Such a demister is a special construction and is not commercially available. In addition its satisfactory operation depends on specific gas flow velocities, which enable the liquid droplets to drop off against the direction of the gas flow. This measure therefore cannot be used directly under varying operating conditions, for example varying flow conditions.

There was therefore in addition a need for a simple measure to prevent solids depositions in the parts of the plant involved in the separation of the aromatic monohydroxy compound from bis(4-hydroxyaryl)alkanes, such as for example heat exchangers for the inert gas preheating, as well as in the demisters used for the gas purification, which does not have the aforementioned disadvantages and permits an interference-free continuous operation for as long as possible, so that production downtimes due to blockages can be prevented.

### EMBODIMENTS OF THE INVENTION

An embodiment of the present invention is a process for producing bis(4-hydroxyaryl)alkanes from adducts of bis(4-hydroxyaryl)alkanes and aromatic monohydroxy compounds comprising: a) passing an inert gas through a melt of an adduct of a bis(4-hydroxyaryl)alkane and an aromatic monohydroxy compound at 150° C. to 230° C., wherein said inert gas stream removes said aromatic monohydroxy compound from said melt; b) removing said aromatic monohydroxy compound from said inert gas by condensation; c) compressing, purifying, and passing said inert gas through at least one demister that is charged with a liquid; and d) recycling said inert gas to a).

Another embodiment of the present invention is the above process, wherein said liquid in c) is water or an alkaline aqueous solution.

Another embodiment of the present invention is the above process, wherein said liquid in c) has a pH value in the range of from 7 to 12.

Another embodiment of the present invention is the above process, wherein said liquid in c) is employed in amounts of 1 litre of liquid per 1 to 500 m³ of inert gas.

Another embodiment of the present invention is the above process, wherein said demister in c) is charged on the inflowing side with the liquid.

Another embodiment of the present invention is the above process, wherein said inert gas is preheated before being recycled to a).

Another embodiment of the present invention is the above process, wherein said bis(4-hydroxyaryl)alkanes are of general formula (I):

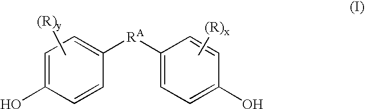

wherein R^{A} is a linear or branched C₁-C₁₈-alkylene radical or a C₅-C₁₈-cycloalkylene radical; R is, independently, a linear or branched C₁-C₁₈-alkyl radical, a C₅-C₁₈-cycloalkyl radical, a C₆-C₂₄-aryl radical, or a halogen radical; and x and y are, independently of one another, 0 or a whole number from 1 to 4.

Another embodiment of the present invention is the above process, wherein R^{A} is a linear or branched C₁-C₆-alkylene radical, or a C₅-C₁₂-cycloalkylene radical; R is, independently, a linear or branched C₁-C₆-alkyl radical, a C₅-C₁₂-cycloalkyl radical, a C₆-C₁₂-aryl radical, or a halogen radical; and x and y are, independently of one another, 0, 1 or 2.

Another embodiment of the present invention is the above process, wherein said aromatic monohydroxy compounds are of general formula (II):

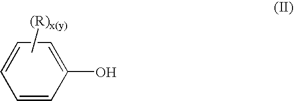

wherein R is, independently, a linear or branched C₁-C₁₈-alkyl radical, a C₅-C₁₈-cycloalkylradical, a C₆-C₂₄-aryl radical, or a halogen radical; and x or y is 0 or a whole number from 1 to 4; with the proviso that said aromatic monohydroxy compounds of general formula (II) are unsubstituted para to the hydroxy group.

Another embodiment of the present invention is the above process, wherein R is, independently, a linear or branched C₁-C₆-alkyl radical, a C₅-C₁₂-cycloalkyl radical, a C₆-C₁₂-aryl radical, or a halogen radical; and x or y is 0, 1 or 2.

Another embodiment of the present invention is the above process, wherein said adduct of a bis(4-hydroxyaryl)alkane and an aromatic monohydroxy compound is the adduct of bisphenol A ((2,2-bis-(4-hydroxyphenyl)-propane) and phenol.

Yet another embodiment of the present invention is a bis(4-hydroxyaryl)alkane with a content of aromatic monohydroxy compounds of less than 100 ppm prepared by the above process.

### DESCRIPTION OF THE INVENTION

The object of the present invention was accordingly to provide such measures.

It is advantageous for the simplest possible measures to employ in this connection a conventional demister that can be used under varying operating conditions.

It was surprisingly found that depositions on and/or blockages of the demister and of the downstream connected parts of the plant can be prevented very efficiently if the commercially available demister in the properly controlled operation of the plant is charged continuously on the inflow side with a wash solution that is removed from the gas stream. In this way the deposition of solids and thus the danger of blockages of the demister can not only be avoided very efficiently but also already formed deposits can possibly be washed off.

The present invention accordingly provides a process for the production of bis(4-hydroxyaryl)alkanes from adducts of bis(4-hydroxyaryl)alkanes and aromatic monohydroxy compounds, in which
a) an inert gas is passed through a melt of an adduct of a bis(4-hydroxyaryl)alkane and an aromatic monohydroxy compound at 150° C. to 230° C., wherein the inert gas stream removes the aromatic monohydroxy compound from the melt,
b) the aromatic monohydroxy compound is removed from the inert gas stream by condensation,
c) the inert gas stream
aa) is compressed, purified and
bb) is passed through at least one demister that is charged with a liquid, and
cc) is recycled to step a).

The process according to the invention can be used to work up the adducts of bis(4-hydroxyaryl)alkanes and aromatic monohydroxy compounds.

Suitable bis(4-hydroxyaryl)alkanes are for example those of the general formula (I),

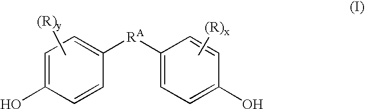

wherein
R^{A} denotes a linear or branched C₁-C₁₈-alkylene radical, preferably C₁-C₆-alkylene radical, or a C₅-C₁₈-cycloalkylene radical, preferably a C₅-C₁₂-cycloalkylene radical,
R independently of one another denotes a linear or branched C₁-C₁₈-alkyl radical, preferably C₁-C₆-alkyl radical, a C₅-C₁₈-cycloalkyl radical, preferably a C₅-C₁₂-cycloalkyl radical, a C₆-C₂₄-aryl radical, preferably a C₆-C₁₂-aryl radical, or a halogen radical, and
x and y independently of one another denote 0 or a whole number from 1 to 4, preferably independently of one another denote 0, 1 or 2.

Preferred bis(4-hydroxyaryl)alkanes are 2,2-bis-(4-hydroxyphenyl)propane (bisphenol A (BPA)), 2,4-bis-(4-hydroxyphenyl)-2-methylbutane, 2,2-bis-(3-methyl-4-hydroxyphenyl)-propane, bis-(3,5-dimethyl-4-hydroxyphenyl)-methane, 2,2-bis-(3,5-dimethyl-4-hydroxyphenyl)-propane, 2,4-bis-(3,5-dimethyl-4-hydroxyphenyl)-2-methylbutane, 1,1-bis-(4-hydroxyphenyl)-cyclohexane and 1,1-bis-(4-hydroxyphenyl)-3,3,5-trimethylcyclohexane (bisphenol TMC).

Particularly preferred bis(4-hydroxyaryl)alkanes are 2,2-bis-(4-hydroxyphenyl)-propane (bisphenol A (BPA)), 2,2-bis(3,5-dimethyl-4-hydroxyphenyl)-propane, 1,1-bis(4-hydroxyphenyl)-cyclohexane and 1,1-bis-(4-hydroxyphenyl)-3,3,5-trimethylcyclohexane (bisphenol TMC).

Most particularly preferred is 2,2-bis-(4-hydroxyphenyl)-propane.

Bis(4-hydroxyaryl)alkanes can be obtained by processes known to the person skilled in the art, by reacting aromatic monohydroxy compounds that are not substituted in the p-position, with ketones that comprise at least one aliphatic group on the carbonyl function.

Suitable aromatic monohydroxy compounds are for example those of the general formula (II),

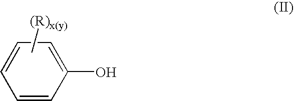

that are not substituted in the p-position, and wherein
R independently of one another denotes a linear or branched C₁-C₁₈-alkyl radical, preferably C₁-C₆-alkyl radical, a C₅-C₁₈-cycloalkylradical, preferably a C₅-C₁₂-cycloalkyl radical, a C₆-C₂₄-aryl radical, preferably a C₆-C₁₂-aryl radical, or a halogen radical, and
x or y denotes 0 or a whole number from 1 to 4, preferably 0, 1 or 2.

Examples of suitable aromatic monohydroxy compounds of the general formula (II) are for example phenol, o- and m-cresol, 2,6-dimethylphenol, o-tert.-butylphenol, 2-methyl-6-tert.-butylphenol, o-cyclohexylphenol, o-phenyl-phenyl, o-isopropylphenol, 2-methyl-6-cyclopentylphenyl, o- and m-chlorophenol or 2,3,6-trimethylphenol. Preferred are phenol, o- and m-cresol, 2,6-dimethylphenol, o-tert.-butylphenol and o-phenyl-phenol, and most particularly preferred is phenol.

Suitable ketones are for example those of the general formula (III),

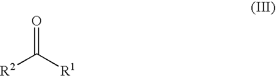

wherein
R¹ denotes a linear or branched C₁-C₁₈-alkyl radical, preferably C₁-C₆-alkyl radical and
R² denotes a linear or branched C₁-C₁₈-alkyl radical, preferably C₁-C₆-alkyl radical, or a C₆-C₂₄-aryl radical, preferably a C₆-C₁₂-aryl radical, or
R¹ and R² together denote a linear or branched C₄-C₁₈-alkylene radical, preferably C₄-C₁₂-alkylene radical.

Examples of suitable ketones of the general formula (III) are acetone, methyl ethyl ketone, methyl propyl ketone, methyl isopropyl ketone, diethyl ketone, acetophenone, cyclohexanone, cyclopentanone, methyl-, dimethyl- and trimethylcyclohexanones, which can also comprise geminal methyl groups, for example 3,3-dimethyl-5-methylcyclohexanone (hydroisophorone). Preferred ketones are acetone, acetophenone, cyclohexanone and their homologues carrying methyl groups; particularly preferred is acetone.

C₁-C₆-alkyl denotes for example methyl, ethyl, n-propyl, iso-propyl, n-butyl, sec.-butyl, tert.-butyl, n-pentyl, 1-methylbutyl, 2-methylbutyl, 3-methylbutyl, neo-pentyl, 1-ethylpropyl, cyclohexyl, cyclopentyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl or 1-ethyl-2-methylpropyl, C₁-C₁₈-alkyl in addition denotes for example n-heptyl and n-octyl, pinacyl, adamantyl, the isomeric menthyls, n-nonyl, n-decyl, n-dodecyl, n-tridecyl, n-tetradecyl, n-hexadecyl, n-octadecyl or stearyl.

C₁-C₆-alkylene and C₁-C₁₈-alkylene denotes for example the alkylene groups corresponding to the aforementioned alkyl groups.

C₅-C₁₂-cycloalkyl denotes for example cyclopentyl, cyclohexyl, cyclooctyl or cyclododecyl.

Examples of C₆-C₂₄-aryl or C₆-C₁₂-aryl are phenyl, o-, p-, m-tolyl, naphthyl, phenanthrenyl, anthracenyl or fluorenyl.

Halogen can denote fluorine, chlorine, bromine or iodine, preferably fluorine or chlorine, and particularly preferably chlorine.

The adducts of a bis(4-hydroxyaryl)alkane and an aromatic monohydroxy compound used in the process according to the invention contain, as aromatic monohydroxy compound, the already mentioned compounds of the general formula (II).

In a preferred embodiment of the process according to the invention, as adduct of a bis(4-hydroxyaryl)alkane and an aromatic monohydroxy compound, there is used the adduct of bisphenol A (2,2-bis-(4-hydroxyphenyl)-propane) and phenol.

The separation of the aromatic monohydroxy compound from the bis(4-hydroxyaryl)alkane from the melt consisting of the adduct of bis(4-hydroxyaryl)alkane and aromatic monohydroxy compound is carried out at temperatures from 150° C. to 230° C., preferably from 170° C. to 210° C. In this connection an inert gas or steam for example is passed through the melt of the adduct formed form a bis(4-hydroxyaryl)alkane and an aromatic monohydroxy compound, whereby the inert gas stream expels the aromatic monohydroxy compound from the melt. This procedure is hereinafter also termed stripping. Suitable as inert gas are for example nitrogen, carbon dioxide or noble gases, such as for example argon. The preferred inert gas is nitrogen. Aqueous steam for example is suitable as steam. The ratio of inert gas or steam to the amount of the adduct melt is preferably 10 m³ to 1000 m³ of inert gas per tonne of adduct melt. The stripping can if necessary be facilitated by reducing the pressure to between 0.1 and 1 bar_{abs}. in the unit used for the separation of the aromatic monohydroxy compound, though the stripping is preferably carried out under normal pressure or slightly increased pressure, such as for example from 1 to 2 bar_{abs}. The stripping of the aromatic monohydroxy compound with the inert gas is carried out in known types of apparatus, for example in a filled column or packed column. The aromatic monohydroxy compound is removed from the inert gas stream by condensation, for example on a heat exchanger.

In preferred embodiments the separation of the aromatic monohydroxy compound from the bis(4-hydroxyaryl)alkane from the melt consisting of the adduct of bis(4-hydroxyaryl)alkane and aromatic monohydroxy compound is carried out in at least one desorber. In this connection the aromatic monohydroxy compound is expelled with the inert gas stream and thereby separated, and the bis(4-hydroxyaryl)alkane is obtained as bottom product of the desorber. Suitable desorbers are any appropriate desorbtion apparatuses known to the person skilled in the art. Shell-and-tube desorbers and column desorbers (packed desorbers) for example may be mentioned in this connection. The first-mentioned type preferably basically contains shell-and-tube heat exchangers. These shell-and-tube heat exchangers are in preferred embodiments arranged vertically, and in their lower part are equipped with a number of nozzles through which the optionally preheated hot inert gas, preferably at a temperature of 160° C. to 210° C., is fed. The shell-and-tube heat exchangers are heated through tubes and by the outer jacket. The intermediate spaces between the heat exchanger tubes are preferably filled on the product side with ceramic spheres (steatite). The product space of the desorber is largely filled with liquid (melt). Such desorbers are described for example in EP-A 1 242 349.

The inert gas stream extracted from the desorber is if necessary cooled, preferably to 30° to 80° C., and optionally subjected to filtration. The inert gas stream is then compressed in at least one compressor, the stream preferably being compressed to 1.5 to 4 bar_{abs}. Suitable compressors are commercially available and known to the person skilled in the art.

The cycled inert gas is purified by intensive washing (scrubbing) with at least one wash medium. This washing can be carried out for example in a gas scrubber. The wash procedure can, depending on requirements, be carried out once or several times, and in the latter case then optionally in several scrubbers. Suitable gas scrubbers are known to the person skilled in the art, for example wash towers. Suitable as wash medium are liquids that can dissolve or bind the aromatic monohydroxy compounds and thus remove them from the inert gas stream.

In a preferred embodiment, in the compression of the inert gas of the circulating stream in a compressor the sealing liquid employed there is utilised as wash medium. The sealing liquid is a liquid that ensures the lubrication of the rotating mechanical seal(s) of the compressor or compressors.

Suitable wash media are for example water and/or alkaline aqueous solutions. In this connection, within the scope of the invention an alkaline aqueous solution is understood to be a solution with a pH of 7 or more. Water or a weakly alkaline aqueous solution are preferably used as wash medium. The water used as wash medium is preferably demineralised water. The pH value of an employed alkaline aqueous solution is preferably in the range from 7 to 12, more preferably 7.5 to 11, and most particularly preferably 8 to 10. All basically acting, water-soluble substances can be used to prepare a suitable alkaline aqueous solution. Alkali metal hydroxides and alkaline earth metal hydroxides are preferably used for the preparation of a suitable alkaline aqueous solution. In a preferred embodiment of the process according to the invention the pH value is checked before and after the washing so that excessive metering in and insufficient metering in are avoided in the cleaning of the inert gas stream. The inert gas stream can then if necessary be passed to a second wash stage using a second wash medium. The second wash medium then preferably has a pH value from 5 to 7. A particularly preferred second wash medium is water, most particularly preferred being demineralised water.

Unless otherwise stated, it is understood that the specified pH values are measured at 25° C.

For the charging of the demister(s) connected downstream of the compression and purification of the inert gas stream, as liquids there can be used the same wash media as those also used in the first as well as optionally in the second gas scrubbing. Designs of suitable demisters are known to the person skilled in the art and are described in the literature; see for example A. Bürkholz, E. Muschelknautz, Chemie Ingenieur Technik, 1972, Vol. 54, pp. 892-900. Suitable demisters preferably comprise wire mesh packings of metals or plastics. Suitable metals for this purpose include for example stainless steels, aluminium, copper, nickel, while suitable plastics are for example polyethylene, polypropylene or polytetrafluoroethylene.

The charging of the demister with liquid can take place in cocurrent flow, i.e. on the packing side into which the inert gas stream flows, or in counter-current flow, i.e. on the packing side from which the inert gas stream leaves. For counter-current charging a vertical arrangement of the demister with the inert gas stream flowing into the packing from underneath is advantageous. The cocurrent charging can be carried out with any suitable arrangements of the demister. Accordingly, it is therefore preferred to charge the demister with liquid on the packing side into which the inert gas stream flows (cocurrent charging). The charging of the demister with liquid is preferably effected with the aid of nozzles, particularly preferably spray nozzles.

Demineralised water or dilute sodium hydroxide solution is preferably used for the charging of the demister. Sodium hydroxide solution with an NaOH content of up to 10 wt. % is for example suitable as dilute sodium hydroxide solution. Preferably amounts of 1 litre of injected liquid per 1-500 m³ of recycled inert gas, particularly preferably 1 litre of injected wash solution per 30-100 m³ of recycled inert gas, are employed.

Due to this feature of charging the demister with a liquid, the operating time of a demister between two necessary cleaning steps can be increased by a factor of 2 to 500 compared to the conventional operating time of the same demister between two necessary cleaning steps that is not charged with wash solution as described above, without having to interrupt the continuous operation and take into account production downtimes.

The inert gas stream cleaned in this way is recycled to step a) of the process according to the invention. Thus, the employed inert gas is cycled in the process according to the invention, which makes the process particularly efficient and economical.

Preferably the recycled inert gas stream can be preheated in an inert gas preheater before being recycled to step a), for example before being recycled to a desorber. This preheating is preferably carried out at temperatures from 150° to 250° C., particularly preferably 170° to 220° C. Heat exchangers, such as for example shell-and-tube heat exchangers or plate heat exchangers, are suitable as inert gas preheaters. Suitable heat exchangers are described in the literature and known to the person skilled in the art.

Deposits and thus blockages of the optionally used inert gas preheaters are significantly reduced or almost completely avoided by the process according to the invention, and thus the number of necessary cleanings, which require an interruption of the continuous operation and involve production downtimes, are significantly reduced.

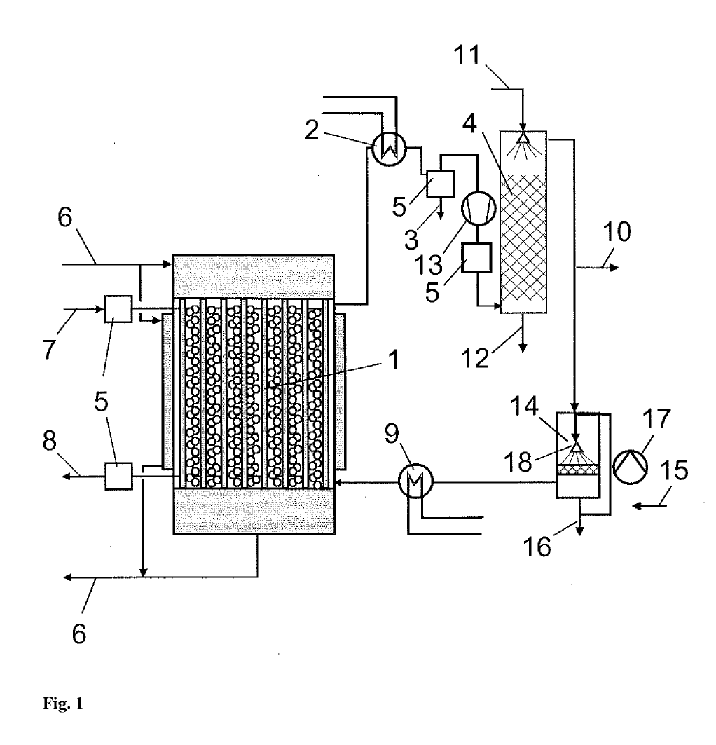

FIG. 1 illustrates by way of example a particularly preferred embodiment of the process according to the invention, without however being restricted thereto. In the FIGURE the reference numerals denote the following items:

1 Desorber
2 Cooler
3 Aromatic monohydroxy compound outflow
4 Gas wash tower (gas scrubber)
5 Filter
6 Heating
7 Adduct feed unit
8 Bis(4-hydroxyaryl)alkane product removal unit
9 Inert gas heater (heat exchanger)
10 Gas discharge
11 Gas wash water feed
12 Gas wash water discharge
13 Circulating gas compressor
14 Demister
15 Demister wash liquid feed
16 Demister wash liquid discharge
17 Demister wash liquid pump
18 Spray nozzle

The adduct of bis(4-hydroxyaryl)alkane and aromatic monohydroxy compound, preferably of bisphenol A and phenol, is fed in through the adduct feed unit (7) via a filter (5) to the desorber (1). The desorber is a shell-and-tube heat exchanger, whose interstices between the heat exchanger tubes on the product side are filled with ceramics spheres (steatite). The desorber is heated by the heating means (6) in order to melt the adduct, and the bis(4-hydroxyaryl)alkane, preferably bisphenol A, obtained in the lower part (bottom of the desorber) is discharged from the desorber through a filter (5) and then through the product removal unit (8). The inert gas is introduced through an inert gas heater (9) into the lower part of the desorber and passed through the adduct melt. The inert gas stream charged with the aromatic monohydroxy compound, preferably phenol, is discharged from the upper part of the desorber, cooled in a cooler (2), and passed through a filter (5). During the cooling the major proportion of the aromatic monohydroxy compound contained in the inert gas stream is condensed out. The aromatic monohydroxy compound, preferably phenol, thereby removed from the inert gas is discharged through an outlet (3). The inert gas stream is then compressed in the compressor (13) and led through a further filter (5) into the lower part of a gas wash tower, into which gas wash water is introduced through a feed line (11) and gas wash water charged with impurities, for example aromatic monohydroxy compound, is removed via a discharge line (12). The inert gas washed in this way is extracted in the upper part of the gas wash tower (4) and passed through the demister (14), which is charged, i.e. sprayed, with liquid (demister wash liquid) by means of a spray nozzle (18) on the side into which the inert gas stream flows. In this connection uncharged demister wash liquid is added from outside through a feed line (15) and led via a pump (17) into the nozzle (18), and the demister wash liquid charged with impurities, for example aromatic monohydroxy compound, is removed completely or in part through a discharge line (16). The demister wash liquid can within the scope of the invention be completely replaced by fresh, uncharged wash liquid through the feed line (15), and the charged wash liquid can be removed completely through the discharge line (16). It is however also possible to mix part of the charged demister wash liquid with added fresh, uncharged wash liquid through the feed line (15) and thereby to some extent use it repeatedly. In this connection only part of the charged wash liquid is removed through the discharge line (16). Gaseous secondary components are removed from the cycle through the gas discharge line (10), thereby preventing such secondary components from accumulating above a certain concentration.

The bis(4-hydroxyaryl)alkanes produced by the process according to the invention are characterised by a very good intrinsic colour as well as a high purity, and in particular they contain only minor amounts of aromatic monohydroxy compounds of preferably less than 100 ppm, particularly preferably less than 50 ppm, and also minor amounts of decomposition products, such as for example isopropenylphenol and dimeric isopropenylphenol.

Within the scope of the present invention ppm are understood to refer to parts by weight, unless otherwise stated.

Polymers such as polycarbonates or epoxy resins with low intrinsic colour can be produced from the bis(4-hydroxyaryl)alkanes produced by the process according to the invention.

The following examples serve to illustrate the invention and should not be regarded as a limitation.

All the references described above are incorporated herein by in their entireties for all useful purposes.

While there is shown and described certain specific structures embodying the invention, it will be manifest to those skilled in the art that various modifications and rearrangements of the parts may be made without departing from the spirit and scope of the underlying inventive concept and that the same is not limited to the particular forms herein shown and described.

### EXAMPLES

### Example 1

39 tonnes/hour of melt mixture consisting of BPA/phenol (90/10 wt. %) are introduced in parallel into five desorbers. A total of 5840 m³ of nitrogen/hour (cycle stream) is continuously introduced into the desorbers. The phenol is taken up by the nitrogen and is then condensed by means of several heat exchangers. The nitrogen is then fed to three compressors, the sealing liquid of which is charged with dilute NaOH solution (pH 10), compressed, and introduced into a gas washer that is operated with demineralised water. The nitrogen treated in this way is passed through a demister consisting of a metal packing (stainless steel mesh packing of stainless steel 1.4571), wherein 100 litres/hour of demineralised water are distributed on the inflow side of the demister over the surface of the demister through a fully spherical nozzle. The inert gas is then, after preheating to 190° C. in the heat exchangers (one nitrogen preheater per desorber), recycled to the desorber and used for the phenol stripping. A low phenol content (phenol value 40 ppm) of pale-coloured BPA melt with a melt colour index of 8 Hazen is thereby obtained. The operational life of the demister is more than three months, and deposits on the nitrogen preheaters are almost completely avoided.

### Comparative Example 1

The implementation is carried out as in Example 1, with the difference that the injection of water onto the inflow side of the demister is dispensed with. The operational life of the demister is less than 1 day, on account of solids deposits on the metal packing; deposits on the nitrogen preheaters lead to partial blockages after ca. 12 months, with the result that the preheaters have to be taken out of service and cleaned.

1. A process for producing bis(4-hydroxyaryl)alkanes from adducts of bis(4-hydroxyaryl)alkanes and aromatic monohydroxy compounds comprising:
a) passing an inert gas through a melt of an adduct of a bis(4-hydroxyaryl)alkane and an aromatic monohydroxy compound at 150° C. to 230° C., wherein said inert gas stream removes said aromatic monohydroxy compound from said melt;
b) removing said aromatic monohydroxy compound from said inert gas by condensation;
c) compressing, purifying, and passing said inert gas through at least one demister that is charged with a liquid; and
d) recycling said inert gas to a).

2. The process of claim 1, wherein said liquid in c) is water or an alkaline aqueous solution.

3. The process of claim 1, wherein said liquid in c) has a pH value in the range of from 7 to 12.

4. The process of claim 1, wherein said liquid in c) is employed in amounts of 1 litre of liquid per 1 to 500 m³ of inert gas.

5. The process of claim 1, wherein said demister in c) is charged on the inflowing side with the liquid.

6. The process of claim 1, wherein said inert gas is preheated before being recycled to a).

7. The process of claim 1, wherein said bis(4-hydroxyaryl)alkanes are of general formula (I): 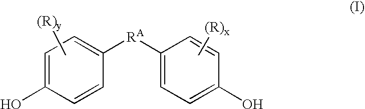
wherein
R^{A} is a linear or branched C₁-C₁₈-alkylene radical or a C₅-C₁₈-cycloalkylene radical;
R is, independently, a linear or branched C₁-C₁₈-alkyl radical, a C₅-C₁₈-cycloalkyl radical, a C₆-C₂₄-aryl radical, or a halogen radical; and
x and y are, independently of one another, 0 or a whole number from 1 to 4.

8. The process of claim 7, wherein:
R^{A} is a linear or branched C₁-C₆-alkylene radical, or a C₅-C₁₂-cycloalkylene radical;
R is, independently, a linear or branched C₁-C₆-alkyl radical, a C₅-C₁₂-cycloalkyl radical, a C₆-C₁₂-aryl radical, or a halogen radical; and
x and y are, independently of one another, 0, 1 or 2.

9. The process of claim 1, wherein said aromatic monohydroxy compounds are of general formula (II): 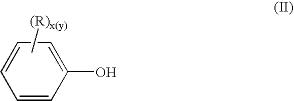
wherein
R is, independently, a linear or branched C₁-C₁₈-alkyl radical, a C₅-C₁₈-cycloalkylradical, a C₆-C₂₄-aryl radical, or a halogen radical; and
x or y is 0 or a whole number from 1 to 4;
with the proviso that said aromatic monohydroxy compounds of general formula (II) are unsubstituted para to the hydroxy group.

10. The process of claim 9, wherein:
R is, independently, a linear or branched C₁-C₆-alkyl radical, a C₅-C₁₂-cycloalkyl radical, a C₆-C₁₂-aryl radical, or a halogen radical; and
x or y is 0, 1 or 2.

11. The process of claim 1, wherein said adduct of a bis(4-hydroxyaryl)alkane and an aromatic monohydroxy compound is the adduct of bisphenol A ((2,2-bis-(4-hydroxyphenyl)-propane) and phenol.

12. A bis(4-hydroxyaryl)alkane with a content of aromatic monohydroxy compounds of less than 100 ppm prepared by the process of claim 1.
